(19) United States
(12) Patent Application Publication
(10) Pub. No.: US 2005/0176121 A1
(43) Pub. Date: Aug. 11, 2005

(54) Method for producing alcohol by using microorganism

(76) Inventors: Ryo Takeshita, Kawasaki-shi (JP); Hisashi Yasueda, Kawasaki-shi (JP); Yoshiya Gunji, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 10/791,853

(22) Filed: Mar. 4, 2004

Related U.S. Application Data
(63) Continuation of application No. PCT/JP02/09029, filed on Sep. 5, 2002.

(30) Foreign Application Priority Data
Sep. 6, 2001 (JP) 2001-270903

(51) Int. Cl. C12P 7/02; C12N 9/00

(52) U.S. Cl. 435/155; 435/170; 435/183

(57) ABSTRACT

The present invention describes a recombinant of a microorganism that does not inherently utilize an alkane, and an alcohol, whereby the recombinant has acquired an ability to convert the alkane into the alcohol due to transformation with a DNA encoding a methane oxygenase. The present invention describes a method for producing alcohol by culturing the recombinant, and allowing the obtained culture, cells isolated from the culture or processed product of the cells to exist with the alkane to produce the alcohol.

This application is a continuation of application PCT/JP02/09029, filed Sep. 5, 2002. All documents cited herein, as well as the foreign priority document, JP 2001-270903, filed Sep. 6, 2001, are hereby incorporated by reference.

### BACKGROUND OF THE INVENTION

1. Field of the Invention

The present invention relates to a method for producing an alcohol such as methanol by utilizing a microorganism. More specifically, the present invention relates to a method for converting an alkane into an alcohol under extremely mild conditions of biochemical oxidation using a microorganism.

2. Description of the Related Art

The currently used process for producing methanol comprises a “synthesis gas production step” for reforming methane as the raw material into a mixed gas of carbon monoxide and hydrogen, and a “methanol synthesis step” for reacting the synthesis gas in the presence of a catalyst to convert the gas into methanol. The synthesis gas production step and the methanol synthesis step are carried out at a high temperature and high pressure, i.e., the synthesis gas production step is carried out at 850 to 880° C., and the methanol synthesis step is carried out at 50 to 100 atm. Therefore, a methanol synthesis method that can be carried out under milder conditions is desirable, and has been studied. Although a method of synthesizing methanol from methane with high yield by using a comparatively thermostable metal complex-type catalyst has been reported recently (Science, Vol. 280, 24, 560-563, April, 1998), this method still requires a temperature of 100° C. or higher.

Meanwhile, if a methane monooxygenase enzyme (hereinafter abbreviated as “MMO”) of a methane-utilizing bacterium is used, methanol can be directly synthesized from methane at an ordinary temperature and ordinary pressure. It is known that there is a soluble-type MMO and a membrane-bound-type MMO. The soluble-type MMO enzyme is a complex protein consisting of three components: hydroxylase, Component B and reductase (Component C). The oxidation reaction is carried out by the hydroxylase with the components, and it has been reported that if this hydroxylase is chemically reduced, instead of using a reductase, the oxidation reaction can be catalyzed by the hydroxylase alone (J. Biol. Chem., 264 (17) 10023-10033, 1989). The hydroxylase is also called Component A, and consists of three kinds of protein subunits: α, β and γ.

U.S. Pat. No. 5,190,870 discloses an enzymatic method for producing an alkanol utilizing a hydroxylase purified from a methane-utilizing bacterium. However, the method for purifying this hydroxylase is complicated, and a decrease in the activity of the enzyme is significant after isolation thereof, which results in an unstable activity of the enzyme. Furthermore, although U.S. Pat. No. 5,192,672 discloses a method for stabilizing an activity of hydroxylase after purification, the fundamental problem of the complicated purification method is still unsolved.

Furthermore, a method of microbiologically producing methanol using a methane-utilizing bacterium itself is also known. However, cells having MMO also have a methanol dehydrogenase etc., and therefore the problem exists that methanol produced by the oxidation of methane is immediately oxidized and thereby converted into formaldehyde, i.e., the methanol is metabolized in the cells. Japanese Patent Laid-open (KOKAI) No. 3-43090 discloses a method of selectively inhibiting a methanol dehydrogenase with cyclopropane. However, the method is extremely complicated, i.e., it comprises substitution of cyclopropane for aerial phase in a suspension of cells containing MMO, subsequent removal of the cyclopropane with helium etc., and the method also suffers from the problem that the methanol dehydrogenase may not be sufficiently inactivated with cyclopropane.

In the Journal of General Microbiology (1992), 138, 1301-1307, it is described that MMO activity could be successfully expressed by expressing Component B and reductase of the soluble-type MMO derived from Methylococcus capsulatus (Bath) in Escherichia coli and mixing them with a natural hydroxylase. Furthermore, in Arch. Microbiol. (1999) 171:364-370, it is reported that the MMO activity could be successfully expressed by introducing a gene of soluble-type MMO into a methane-utilizing bacterium having only the membrane-bound-type MMO. However, there is no example of expressing the activities of all of the components of MMO in a microorganism that does not utilize methane or methanol, and therefore, it is desirable to express the complicated enzyme complex in a heterogenous organism.

### SUMMARY OF THE INVENTION

It is an object of the present invention to express all of the components of soluble-type MMO (henceforth also abbreviated as “sMMO”) in a microorganism that does not utilize methane and methanol, and thereby produce active sMMO, and to provide a method for producing an alcohol from an alkane by utilizing such a microorganism.

It is an object of the present invention to provide a method for producing an alcohol, comprising culturing a recombinant of a microorganism that does not inherently utilize an alkane, and an alcohol which is generated by oxidation of the alkane, which recombinant has acquired an ability to convert the alkane into the alcohol due to transformation with a DNA encoding a methane oxygenase, and allowing the obtained culture, cells isolated from the culture or processed product of the cells to exist with the alkane to produce the alcohol.

It is a further object of the present invention to provide the method as described above, wherein the methane oxygenase is a soluble-type methane oxygenase.

It is a further object of the present invention to provide the method as described above, wherein the methane oxygenase consists of a methane hydroxylase, Component B and reductase.

It is a further object of the present invention to provide the method as described above, wherein the DNA encoding the methane oxygenase is a soluble-type methane oxygenase gene of Methylococcus capsulatus.

It is a further object of the present invention to provide the method as described above, wherein the microorganism is an Escherichia bacterium, coryneform bacterium or Bacillus bacterium.

It is a further object of the present invention to provide the method as described above, wherein the microorganism is an Escherichia bacterium.

It is a further object of the present invention to provide the method as described above, wherein the microorganism is cultured at between 20 to 30° C.

It is a still further object of the present invention to provide the method as described above, wherein the alkane is an alkane having between 1 to 8 carbon atoms, and the alcohol is an alcohol which is generated by oxidation of the alkane.

It is even a further object of the present invention to provide the method as described above, wherein the alkane is methane, and the alcohol is methanol.

According to the present invention, an ability to convert an alkane into an alcohol can be imparted to a microorganism which does not inherently utilize the alkane, and an alcohol which is generated by oxidation of the alkane, and the alcohol can be produced from the alkane using the obtained microorganism.

### BRIEF EXPLANATION OF THE DRAWING

FIG. 1 shows calibration curves prepared by plotting the methanol concentration (mM) in abscissa and the absorbance in ordinate. The results for E. coli JM109/pRS are indicated with ♦, and the results for E. coli JM109/pRSsMMOTB (AJ13852) are indicated with ▪.

### DESCRIPTION OF THE PREFERRED EMBODIMENTS

The inventors of the present invention originally attempted to introduce a gene cluster encoding the soluble-type MMO into a microorganism which lacked any enzyme that oxidizes methanol such as methanol dehydrogenase, i.e., a microorganism that does not utilize methane or methanol, to construct a microorganism that expresses the gene cluster as active proteins and thereby provide a method for producing methanol from methane by utilizing the microorganism. However, sMMO is a complex protein consisting of 5 subunits, and therefore it is not easy to express it in an active form, in particular, in a heterogenous microorganism. Therefore, they conducted various research on types of plasmids and promoters useful for a plasmid for expressing the soluble-type MMO and culture conditions. As a result, they succeeded in expressing sMMO in cells of a heterogenous microorganism while maintaining the activity thereof. Furthermore, they also succeeded in producing methanol from methane by using the obtained microorganism, and thus accomplished the present invention.

Hereinafter, the present invention will be explained in detail.

The microorganism used for the present invention is a microorganism which does not inherently utilize an alkane, and an alcohol which is generated by oxidation of the alkane, but the microorganism has acquired an ability to convert the alkane into the alcohol because it has been transformed with a DNA encoding sMMO. As for the characteristic that the microorganism used for the present invention does not utilize an alcohol, whether the microorganism converts an alkane into an alcohol or not is not essential, whereas it is preferred that the microorganism does not utilize the alcohol for accumulation of the produced alcohol.

The microorganism which does not inherently utilize an alkane, and an alcohol which is generated by oxidation of the alkane, is not particularly limited so long as it is a microorganism that can acquire an ability to convert the alkane to the alcohol. Specific examples include, but are not limited to, Escherichia bacteria such as Escherichia coli, coryneform bacteria such as Brevibacterium lactofermentum (Corynebacterium glutamicum), and Bacillus bacteria such as Bacillus subtilis, and so forth.

The DNA encoding a methane oxygenase is preferably a DNA encoding a soluble-type MMO (sMMO). sMMO consists of Component A (methane hydroxylase), Component B and Component C (reductase). Component A consists of the subunits α, β and γ. Although these components may be separately introduced into the microorganism, they are preferably introduced by using a single vector containing a DNA encoding all of the components. Hereinafter, a gene cluster encoding all of the components of sMMO is referred to as an sMMO gene for convenience.

The sMMO gene can be obtained from a chromosomal DNA of a methane-utilizing bacterium, for example, the Methylococcus capsulatus NCIMB 11132 strain. This strain can be obtained from NCIMB (The National Collections of Industrial, Food and Marine Bacteria Ltd., 23 St. Machar Drive, Aberdeen AB2 1RY, Scotland, UK).

An example of a method for preparing a fragment containing the sMMO gene from Methylococcus capsulatus as a methane-utilizing bacillus is described herein. However, such a fragment can also be obtained from other methane-utilizing bacteria in a similar manner.

First, the medium used for culturing Methylococcus capsulatus may be any medium in which the bacterium can sufficiently proliferate. An example of a preferred medium includes the medium of Whittenbury et al. (J. Gen. Microbiol., 61, 205-208, 1970). Any space in a culture vessel containing the medium is replaced with a mixed gas of methane and an oxygen-containing gas (air etc.), and Methylococcus capsulatus is inoculated into the medium in contact with the gas. Methylococcus capsulatus is an aerobic bacterium, and the culture may be performed at a temperature of between 20 to 50° C. under aerobic conditions as a batch culture or a continuous culture.

A DNA fragment containing the sMMO gene can be separated and obtained by the hybridization method in the manner described below. The source of the DNA fragment can be a DNA library derived from chromosomes of a Methylococcus capsulatus strain using, as a probe, a DNA fragment containing a part of the sMMO gene obtained via PCR (polymerase chain reaction). Oligonucleotides useful as primers are prepared based on the known nucleotide sequence of the sMMO gene (GenBank Accession M90050 M32314 M58498 M58499, Stainthorpe, A. C., et al., Gene, 91 (1), 27-34, 1990, SEQ ID NO: 5), and chromosomal DNA from Methylococcus capsulatus can be used as a template. The open reading frames contained in the sMMO gene are designated mmoX, mmoY, mmoB, mmoZ, OrfY and mmoC in this order from the 5′ end.

The chromosomal DNA can be extracted from the culture broth of the Methylococcus capsulatus NCIMB 11132 strain by a usually used method known per se (e.g., the method described in Biochem. Biophys. Acta., 72, 619 (1963) etc.).

The DNA library can be prepared by digesting the chromosomal DNA using a suitable restriction enzyme such as BamHI, ligating the obtained DNA fragments of various sizes with a plasmid vector such as pUC18 (purchased from TAKARA SHUZO CO., LTD) and transforming a suitable host such as Escherichia coli JM109 with the ligation reaction mixture.

The probe for selecting a clone having a DNA fragment containing the sMMO gene by hybridization can be obtained by PCR using oligonucleotides suitably designed based on the known nucleotide sequence of the sMMO gene, for example, the nucleotide sequences shown as SEQ ID NOS: 1 and 2, as primers and a chromosomal DNA of Methylococcus capsulatus as a template.

Colony hybridization is performed for a chromosomal DNA library of the Methylococcus capsulatus NCIMB 11132 strain by using the probe obtained as described above. A DNA fragment containing the sMMO gene or a part thereof can be obtained by extracting a plasmid DNA from a clone that hybridizes with the partial DNA fragment of the sMMO gene used as the probe in the hybridization and obtaining the inserted fragment through digestion of the plasmid DNA with a suitable restriction enzyme.

When the fragment of the clone obtained as described above contains a part of the sMMO gene, the other region can be obtained by the PCR method, hybridization method or the like. For example, when the cloned fragment does not have the 5′ side region of the sMMO gene, the upstream region of the cloned fragment can be obtained by the 5′-RACE method. Moreover, the upstream region of the sMMO gene can also be obtained by PCR using the oligonucleotides shown as SEQ ID NOS: 3 and 4 as primers and the chromosomal DNA of the NCIMB 11132 strain as a template. The obtained DNA fragment can be ligated to the previously obtained sMMO gene fragment to obtain the full length sMMO gene.

Other than wild-type proteins, each component or subunit of sMMO may have an amino acid sequence which includes substitution, deletion, insertion or addition of one or several amino acid residues, as long as the function of each component or subunit is not degraded. Although the number of “several+ amino acids referred to herein differs depending on position of amino acid residues in a three-dimensional structure of a protein or type of amino acids, it may be preferably between 2 to 10, more preferably between 2 to 5, most between preferably 2 to 3.

The DNA encoding a protein or peptide substantially identical to the sMMO described above include a DNA that is hybridizable with an open reading frame in the nucleotide sequence shown as SEQ ID NO: 4 or a probe that can be produced from the nucleotide sequence under stringent conditions and codes for a protein that can constitute an active sMMO. The aforementioned “stringent conditions” include conditions under which a so-called specific hybrid is formed, and a non-specific hybrid is not formed. It is difficult to clearly express this condition by using any numerical value. However, for example, the stringent conditions include conditions under which DNAs having high homology, for example, DNAs having homology of 50% or more hybridize with each other, but DNAs having homology lower than the above do not hybridize with each other. Alternatively, the stringent conditions include conditions whereby DNAs hybridize with each other at a salt concentration corresponding to typical washing conditions of Southern hybridization, i.e., approximately 1×SSC, 0.1% SDS, preferably 0.1×SSC, 0.1% SDS, at 60° C.

The vector used for introducing the sMMO gene into a host microorganism may be any vector autonomously replicable in a cell of the host microorganism, and specific examples include, for Escherichia coli, plasmid vectors pUC19, pUC18, pBR322, pHSG299, pHSG298, pHSG399, pHSG398, RSF1010, pMW119, pMW118, pMW219, pMW218 and so forth. Furthermore, vectors for coryneform bacteria include pAM330 (refer to Japanese Patent Laid-open Publication No. 58-67699), pHM1519 (refer to Japanese Patent Laid-open Publication No. 58-77895), pAJ655, pAJ611, pAJ1844 (refer to Japanese Patent Laid-open No. 58-192900 for these), pCG1 (refer to Japanese Patent Laid-open No. 57-134500), pCG2 (refer to Japanese Patent Laid-open No. 58-35197), pCG4, pCG11 (refer to Japanese Patent Laid-open No. 57-183799 for these), pHK4 (refer to Japanese Patent Laid-open No. 5-7491) and so forth. Furthermore, vectors for Bacillus bacteria include pUB110, pHY300PLK, pHV1248, pE194, pC194, pBC16, pSA0501, pSA2100, pAM77, pT181, pBD6, pBD8 and pBD64, pHV14 and so forth.

The promoter of the sMMO gene may be replaced with a suitable promoter depending on the microorganism into which the gene is introduced. Examples of such promoters include lac promoter, trp promoter, trc promoter, tac promoter, P_{R} promoter and P_{L} promoter of lambda phage, tet promoter, amyE promoter and so forth. If an expression vector containing a promoter is used as the vector, ligation of the sMMO gene, vector and promoter can be attained by a single ligation operation. Examples of such vectors include pKK233-3 containing the tac promoter (purchased from Pharmacia) and so forth.

Method of transformation for introducing a vector incorporated with the sMMO gene into an objective microorganism include, for example, a method of treating recipient cells with calcium chloride so as to increase the permeability of the cells for DNA, which has been reported for Escherichia coli K-12 (Mandel, M. and Higa, A., J. Mol. Biol., 53, 159 (1970)), and a method of preparing competent cells from cells which are at the growth phase followed by introducing the DNA thereinto, which has been reported for Bacillus subtilis (Duncan, C. H., Wilson, G. A. and Young, F. E., Gene, 1, 153 (1977)) can be used. Alternatively, a method of making DNA-recipient cells into protoplasts or spheroplasts, which can easily take up recombinant DNA, followed by introducing the recombinant DNA into the DNA-acceptor cells, which is known to be applicable to Bacillus subtilis, actinomycetes and yeasts (Chang, S. and Choen, S. N., Molec. Gen. Genet., 168, 111 (1979); Bibb, M. J., Ward, J. M. and Hopwood, O. A., Nature, 274, 398 (1978); Hinnen, A., Hicks, J. B. and Fink, G. R., Proc. Natl. Sci., USA, 75, 1929 (1978)) can also be applicable. The transformation method can be appropriately selected from these methods depending on the cell used as the host. Furthermore, the recombinant DNA can be introduced into a recipient bacterium belonging to Brevibacterium or Corynebacterium by the electroporation method (Sugimoto et al., Japanese Patent Laid-open No. 2-207791) (OP199).

As methods for preparation of genomic DNA library, hybridization, PCR, preparation of plasmid DNA, digestion and ligation of DNA, transformation and so forth, usual methods well known to those skilled in the art can be used. Such methods are described in Sambrook, J., Fritsch, E. F., Maniatis, T., Molecular Cloning, Cold Spring Harbor Laboratory Press, 1.21 (1989).

The E. coli JM109 strain transformed with an expression vector pRSsMMOTB containing the sMMO gene obtained as described above was deposited at the independent administrative agency, National Institute of Advanced Industrial Science and Technology, International Patent Organism Depository (postal code 305-5466, Tsukuba Central 6, 1-1 Higashi 1-Chome, Tsukuba-shi, Ibaraki-ken, Japan) on Aug. 2, 2001 and given an accession number of FERM P-18446. Then, the deposit was converted into an international deposit under the provisions of the Budapest Treaty on Aug. 19, 2002 and received an accession number of FERM BP-8153.

Culturing a microorganism which acquired an ability to convert an alkane into an alcohol by transformation with a DNA encoding the sMMO gene, and allowing the obtained culture or cells isolated from the culture to exist with the alkane results in production of alcohol.

The medium used for the culture of the microorganism into which the sMMO gene is introduced may be appropriately selected depending on the microorganism used. For example, it may be a typical medium that contains a carbon source, nitrogen source, inorganic ions, and other organic ingredients as required.

As the carbon source, saccharides such as glucose, sucrose, lactose, galactose, fructose or starch hydrolysate, alcohols such as glycerol or sorbitol, or organic acids such as fumaric acid, citric acid or succinic acid can be used.

As the nitrogen source, inorganic ammonium salts such as ammonium sulfate, ammonium chloride or ammonium phosphate, organic nitrogen such as soybean protein hydrolysate, ammonia gas, aqueous ammonia and so forth can be used.

It is desirable to add required substances such as vitamin B₁, yeast extract and so forth to the medium in appropriate amounts as organic trace nutrients. Other than the above, potassium phosphate, magnesium sulfate, iron ions, manganese ions and so forth can be added in small amounts as required.

The culture is preferably carried out under aerobic conditions for between 16 to 72 hours. The culture temperature is preferably controlled to be between 25° C. to 45° C., and pH is preferably controlled to be between 5 to 8 during the culture. Inorganic or organic, acidic or alkaline substances as well as ammonia gas and so forth can be used for adjustment of pH.

When an Escherichia bacterium such as Escherichia coli is used as the host microorganism, the culture is preferably performed at a temperature of between 20 to 30° C. in order to produce the subunits constituting sMMO as soluble peptides.

As the catalyst for conversion of an alkane to an alcohol, besides cells and culture containing cells, a processed product of the cells may also be used. The processed product of cells may be cells treated with acetone, lyophilized cells, cell-free extract prepared from the acetone-treated or lyophilized cells or live cells, fractionation product such as membrane fraction fractionated from the cell-free extract, and immobilized product of the cells, cell-free extract or fractionation product. By bringing such cells or processed product of cells into contact with an alkane and allowing a reaction, an alcohol can be produced in the reaction solution. The microorganism used may be one kind of microorganism or a mixture of two or more kinds of arbitrary microorganisms.

If the alkane does not inhibit growth of the microorganism, the alkane can be added to the medium used for the culture of the microorganism to simultaneously perform the culture of the microorganism and production of the alcohol. In such a case, the alkane can be introduced into the medium by adding it into a gas phase in contact with the medium or bubbling the medium with the alkane. Furthermore, by using a microorganism exhibiting strong reducing power such as Escherichia bacteria as the microorganism used for the present invention or by adding NADH to the reaction mixture, the reaction catalyzed by sMMO can be efficiently advanced.

The alkane of the present invention is preferably an alkane having between 1 to 8 carbon atoms, more preferably between 1 to 6 carbon atoms, and particularly preferably between 1 to 5 carbon atoms. Especially, methane is the most preferred. Furthermore, the position of hydrogen atom in the alkane oxidized by sMMO is not particularly limited, and it may be hydrogen bonding to an end carbon or hydrogen bonding to a carbon to which two or more carbons bond. The alkane may be a linear, branched or cyclic alkane. The alkane may have a substituent such as a halogen.

The substrate specificity of sMMO derived from the Methylococcus capsulatus Bath strain has been studied, and it has been revealed that the strain has an ability to oxidizing alkanes, including at least methane to octane to produce corresponding alcohols.

### EXAMPLES

Hereinafter, the present invention will be explained more specifically with reference to the following examples.

<1> Preparation of Chromosomal DNA Library of Methane-Utilizing Bacterium, Methylococcus capsulatus

A space in a culture vessel containing the medium of Whittenbury et al. (J. Gen. Microbiol., 61, 205-208, 1970) was replaced with a mixed gas of methane and air. The methane-utilizing bacterium, Methylococcus capsulatus NCIMB 11132 strain, was inoculated into the medium in contact with the gas and cultured under aerobic conditions as a batch culture, while gas replacement was continued.

Chromosomal DNAs were extracted from the cells of the Methylococcus capsulatus NCIMB 11132 cultured as described above by the method described in Biochem. Biophys. Acta., 72, 619 (1963). The chromosomal DNAs were completely digested with the restriction enzyme BamHI. The obtained DNA fragments of various sizes were inserted into a plasmid vector pUC18 (purchased from TAKARA SHUZO CO., LTDat the BamHI site. Escherichia coli JM109 was transformed with the obtained recombinant plasmids to prepare a chromosomal DNA library.

### <2> Cloning of sMMO Gene by Colony Hybridization

A clone containing a sMMO gene fragment was selected from the aforementioned chromosomal DNA library by colony hybridization. A hybridization probe was prepared by amplifying the sMMO gene fragment by the PCR method. The nucleotide sequence of the sMMO gene of Methylococcus capsulatus has been reported (Gene, 91, 27-34 (1990)), and the oligonucleotides having the nucleotide sequences shown as SEQ ID NOS: 1 and 2 were synthesized based on that sequence.

The chromosomal DNAs of Methylococcus capsulatus prepared as described above was used as the template, and the aforementioned oligonucleotides were used as primers to perform PCR (reaction conditions: a cycle of reaction steps of denaturation: 94° C. for 10 seconds, an annealing: 55° C. for 30 seconds, and extension: 72° C. for 1 minute and 30 seconds was performed for 30 cycles).

Colony hybridization was performed for the aforementioned chromosomal DNA library using the partial fragment of the sMMO gene amplified as described above as a probe. Labeling of the probe and the hybridization reaction were performed using DIG-High Prime DNA Labeling & Detection Kit I (purchased from Boehringer Mannheim) according to the attached protocol.

Recombinant plasmid DNAs were extracted from the clones that showed positive results for hybridization, and the plasmid DNAs were digested with the restriction enzyme BamHI to confirm the inserted fragments. As a result, in addition to a DNA fragment having a length of about 2.3 kb corresponding to the length of the plasmid pUC18, an inserted DNA fragment having a size of about 6 kb was confirmed.

This inserted DNA was digested with various restriction enzymes to confirm that it contained a large part of the objective sMMO gene. As a result, it became clear that a part of a gene encoding the a subunit constituting sMMO was not included in the aforementioned fragment. The recombinant plasmid containing this inserted DNA fragment of about 6 kbp was designated pUC6K.

<3> Construction of Plasmid Containing sMMO Gene of Full Length

Then, an upstream region of the sMMO gene having a deletion was obtained by PCR as described below. The nucleotide sequences of the synthesized primers are shown in SEQ ID NO: 3 and 4. The chromosomal DNAs of the NCIMB 11132 strain were used as a template together with the aforementioned oligonucleotides as primers to perform PCR (a cycle consisting of reaction steps of denaturation: 98° C. for 10 seconds, annealing: 60° C. for 30 seconds, and extension: 72° C. for 180 seconds was repeated for 30 cycles). The amplified DNA fragment of about 1.5 kbp was digested with the restriction enzymes EcoRI and BamHI and ligated to the multi-cloning site of pUC118. A fragment of about 6 kbp excised from pUC6K with BamHI was incorporated into the BamHIsite of the aforementioned plasmid to obtain a plasmid pUCsMMO containing the sMMO gene of the full length.

<4> Construction of sMMO Gene Expression Plasmid

Then, the sMMO gene region was excised from the aforementioned plasmid pUCsMMO using the restriction enzymes EcoRI and HindIII, and incorporated into an expression vector pKK233-3 (purchased from Pharmacia) downstream from the tac promoter to obtain an expression plasmid pKKsMMO, which was constructed so that transcription from the tac promoter proceeds to the sMMO gene.

The sMMO gene including the tac promoter region derived from pKK3-233 was excised from pKKsMMO by digestion with the restriction enzymes NdeI and DraI, blunt-ended, digested with the restriction enzyme PstI, and then ligated to a blunt-ended broad host spectrum vector pRS (described in International Patent Publication in Japanese (Kohyo) No. 3-501682) to obtain a sMMO expression plasmid pRSsMMOTB.

<5> Confirmation of Expression of sMMO

The AJ13852 strain was inoculated into the LB liquid medium containing 20 μg/ml of streptomycin, precultured overnight at. 25° C., then inoculated into a similarly prepared liquid medium in an amount of 1% (v/v), and cultured at 25° C. as the main culture. During the main culture, when OD₆₆₀ reached 0.6, IPTG (isopropyl-β-D-thiogalactopyranoside) was added to the medium at a final concentration of 1 mM, and the cells were further cultured for 2.5 hours. The cells obtained with the aforementioned culture conditions were disrupted by ultrasonication and fractionated into a soluble fraction and insoluble fraction by centrifugation. Each fraction was analyzed by using antibodies directed to each of the component peptides constituting sMMO. As a result, it was confirmed that all of the subunits, α, β and γ and both of Components B and C of sMMO existed in the soluble fraction.

When all of the aforementioned culture steps were performed at 37° C., the a subunit completely formed inclusion bodies and did not exist in the soluble fraction. Furthermore, most of the β and γ subunits were also insoluble. Moreover, when the culture was performed at 30° C., the amount of the a subunit existing in the soluble fraction became smaller as compared with the case where the culture was performed at 25° C.

<6> Production of Methanol from Methane

The AJ13852 strain was cultured in the same manner as described above by using 100 ml of the medium (temperature was 25° C.), and the cells were collected and then washed with 50 mM potassium phosphate buffer (pH 7.0). Then, the cells were suspended in the same buffer (pH 7.0), and a cell-free extract was obtained by ultrasonication. The total protein concentration of the cell-free extract was adjusted to 15 mg/ml. In a volume of 1 ml, the aforementioned cell-free extract was introduced into a 5-ml volume Alumi-Seal vial (purchased from GL Science Inc.), mixed with NADH (reduced nicotinamide adenine dinucleotide) to a final concentration of 1 mM, and sealed. Then, 5 ml (volume under 1 atm) of methane was enclosed in the vial. The reaction was allowed at 37° C. for 60 minutes with shaking. Furthermore, the AJ13852 strain and E. coli JM109/pRS introduced only with the vector pRS were also cultured in the same manner as described above, except that methane was not enclosed.

The methanol concentration was quantified using a quantification system having three steps of enzymatic reactions. The compositions of reaction mixtures are shown in Table 1. First, 600 μl of the reaction mixture after the aforementioned methanol formation reaction was taken, mixed with 50 μl of 5 N potassium hydroxide, sufficiently stirred and left for 5 minutes. Then, the reaction mixture was mixed with 50 μl of 5 M sodium chloride and adjusted to pH 7.5 with 1 M potassium phosphate buffer. Thereafter, each reaction mixture was used for the following enzymatic reaction after the denatured proteins were removed by centrifugation.

TABLE 1
Calibration curve

| | |
|---|---|
| MeOH (0 to 1000 mM) | 20 μl |
| Oxidized nicotinamide adenine dinucleotide | 2 μl |
| (final concentration: 1 mM) | |
| Alcohol oxidase (1 mg/ml) | 7 μl |
| Formaldehyde dehydrogenase (1 mg/ml) | 17 μl |
| Diaphorase (final concentration: 2 U/ml) | 20 μl |
| Iodonitrotetrazolium (final concentration: 1 mM) | 20 μl |
| Sample (without methane gas) | 20 μl |
| 25 mM Potassium phosphate buffer | 94 μl |
| Total | 200 μl |

Measurement sample

| | |
|---|---|
| Oxidized nicotinamide adenine dinucleotide | 2 μl |
| (final concentration: 1 mM) | |
| Alcohol oxidase (1 mg/ml) | 7 μl |
| Formaldehyde dehydrogenase (1 mg/ml) | 17 μl |
| Diaphorase (final concentration: 2 U/ml) | 20 μl |
| Iodonitrotetrazolium (final concentration: 1 mM) | 20 μl |
| Sample (with methane gas) | 20 μl |
| 25 mM Potassium phosphate buffer | 114 μl |
| Total | 200 μl |

The enzymatic reaction was performed according to the following procedures. Methanol was converted into formaldehyde with the alcohol oxidase, and formic acid was produced from formaldehyde with the formaldehyde dehydrogenase. NADH produced in the above reaction was quantified using the color reaction of the diaphorase as absorbance at 550 nm. The alcohol oxidase and formaldehyde dehydrogenase were purchased from Sigma, diaphorase was purchased from Toyobo, and iodonitrotetrazolium was purchased from Nakalai Tesque.

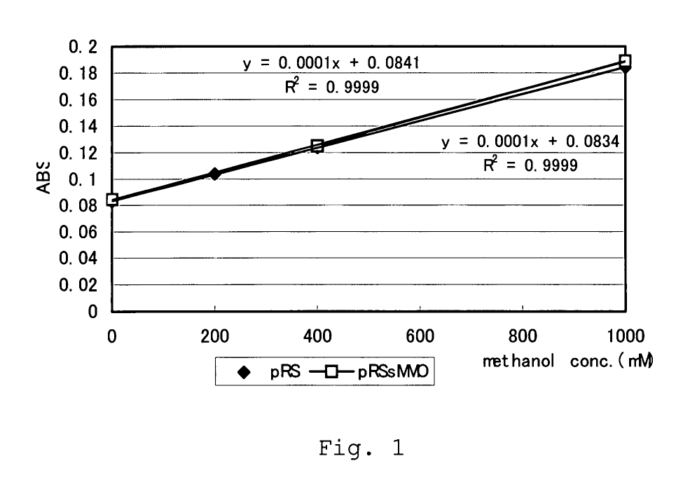

The reaction mixtures obtained using the AJ13852 strain and E. coli JM109/pRS which was introduced only with the vector pRS without enclosing methane were subjected to the aforementioned three-step enzymatic reaction, and the measured absorbance values were considered to correspond to a methanol concentration of 0 M. On the basis of these values, methanol was added to the aforementioned quantification system at various concentrations, and the methanol amounts were plotted against absorbance values to prepare calibration curves (FIG. 1). As a result, it was found that the methanol concentration value linearly corresponded to the absorbance value in the methanol concentration range of 200 to 1000 μM in this measurement system.

The methanol production amount for each kind of cell was quantified using the aforementioned calibration curves. As a result, production of methanol was not detected for E. coli JM109/pRS, whereas production of methanol was detected for the AJ13852 strain, and the concentration was 240 μM. The specific activity in this reaction was 0.33 nmol/minute/1 mg protein in the enzyme solution.

While the invention has been described in detail with reference to preferred embodiments thereof, it will be apparent to one skilled in the art that various changes can be made, and equivalents employed, without departing from the scope of the invention.

1. A method for producing an alcohol comprising
(A) culturing a recombinant of a microorganism that does not inherently utilize an alkane, and an alcohol which is generated by oxidation of an alkane, whereby said recombinant has acquired an ability to convert the alkane into the alcohol due to transformation with a DNA encoding a methane oxygenase, and
(B) allowing the obtained culture, cells isolated from the culture, or processed product of the said cells to exist with the alkane to produce the alcohol.

2. The method for producing an alcohol according to claim 1, wherein said methane oxygenase is a soluble-type methane oxygenase.

3. The method for producing an alcohol according to claim 2, wherein said methane oxygenase comprises a methane hydroxylase, Component B and a reductase.

4. The method for producing an alcohol according to claim 1, wherein said DNA encoding the methane oxygenase is a soluble-type methane oxygenase gene of Methylococcus capsulatus.

5. The method for producing an alcohol according to claim 1, wherein said microorganism is selected from the group consisting of Escherichia bacterium, coryneform bacterium, and Bacillus bacterium.

6. The method for producing an alcohol according to claim 5, wherein said microorganism is an Escherichia bacterium.

7. The method for producing an alcohol according to claim 6, wherein said microorganism is cultured at a temperature of between 20 to 30° C.

8. The method for producing an alcohol according to claim 1, wherein said alkane comprises an alkane having between 1 to 8 carbon atoms, and said alcohol comprises an alcohol which is generated by oxidation of the alkane.

9. The method for producing an alcohol according to claim 8, wherein said alkane is methane, and said alcohol is methanol.
